(19) United States
(12) Patent Application Publication
(10) Pub. No.: US 2015/0164464 A1
(43) Pub. Date: Jun. 18, 2015

(54) DEVICE AND METHOD FOR DERIVING CT NUMBERS IN CONE BEAM COMPUTED TOMOGRAPHY

(71) Applicants: Peter Mah, San Antonio, TX (US); W. Doss McDavid, San Antonio, TX (US)

(72) Inventors: Peter Mah, San Antonio, TX (US); W. Doss McDavid, San Antonio, TX (US)

(73) Assignee: BOARD OF REGENTS OF THE UNIVERSITY OF TEXAS SYSTEM, Austin, TX (US)

(21) Appl. No.: 14/129,326

(22) PCT Filed: Jun. 28, 2012
(86) PCT No.: PCT/US2012/044536

§ 371 Date: Sep. 18, 2014

Related U.S. Application Data
(60) Provisional application No. 61/502,595, filed on Jun. 29, 2011.

(51) Int. Cl. A61B 6/00; A61B 6/03; A61B 6/14

(52) U.S. Cl. CPC A61B 6/583; A61B 6/14; A61B 6/032

(57) ABSTRACT

Described herein is a device and method to derive density measurements for features depicted in computed tomography systems. A radiographic device is used in some embodiments to derive a correlation between measured grey levels in an image produced using a CT system and the attenuation coefficients and/or the CT numbers of the depicted features.

### BACKGROUND OF THE INVENTION

1. Field of the Invention

The invention generally relates to x-ray computer tomography. More specifically the invention relates to the devices and methods for determining bone and tissue CT numbers.

2. Description of the Relevant Art

Cone beam computer tomography (CBCT) systems offer many advantages over medical computer tomography (CT) for dental treatment and planning, including a lower radiation dose to the patient in most instances, shorter acquisition times for the resolution desired in dentistry, an affordable cost alternative to medical CT, better resolution and greater detail. The disadvantages associated with CBCT scanners are: increased scatter radiation, the limited dynamic range of x-ray area detectors, beam hardening artifacts and the inability to display actual CT numbers or Hounsfield units as in medical CT.

For successful implant planning, it is important to be able to assess the bone quality in addition to the height and width of bone and the distance to other anatomical structures such as the mandibular canal or sinus region. Hounsfield units provide a quantitative assessment of bone density as measured by its ability to attenuate an x-ray beam. The displayed grey levels in CBCT systems are arbitrary and do not allow for the assessment of bone quality, which can be performed with Hounsfield units in medical CT.

Hounsfield units provide a standard scheme for scaling the reconstructed attenuation coefficients in medical CT systems. To date, the manufacturers of dental CBCT systems have not used a standard system for scaling the grey levels representing the reconstructed values. In the absence of such a system, it is difficult to interpret the grey levels or to compare the values resulting from different machines. A review of the literature has found that while there is an acknowledgment that a deficiency exists with CBCT systems, they do not correctly display Hounsfield units, there has been little research conducted to correct this deficiency.

For example, in a study it was found that calculated density (Hounsfield units) on a CBCT scan varied widely from a range of −1500 to over +3000 for different types of bone. The researchers concluded that the ability to assess the density or quality of bone is limited and, in regions where it is clearly soft tissue, the Hounsfield units vary greatly and provide little or no meaningful data. The researchers stated “The HU is the standard scale for the measurement of conventional CT values. Without HU, it can be difficult to analyze bone quality and to process and scan through two-dimensional (2D) and three-dimensional (3D) images using various standard DICOM software products with default settings for conventional CT images”.

In another study it was concluded that Hounsfield units sampled from identical anatomic areas with CBCT and MDCT are not identical. Data from CBCT studies is difficult to threshold for anatomic model output since automatic thresholding algorithms are less successful. The result is generally reliance on more manual methods which can be more time consuming and subjective.”

Further studies have concluded that CBCT grey levels are inaccurate to rely upon for decisions on implant placement. For example in one study it was concluded that though CBCT machines also display gray scale units, they are not ‘true’ Hounsfield units. The study found that the values assigned to the pixels (picture elements) are relative Hounsfield units and cannot be used as precisely to estimate bone density. The study concluded that there is no good data to relate Hounsfield units to the quality of bone for a desired implant site, although clinicians place great faith in the Hounsfield units in an attempt to determine whether or not their implant fixture will be placed in ‘good bone’.”

Hounsfield units is the standard of measurement for conventional CT machines to analyze bone quality and process 2D and 3D images with the standard Digital Imaging and Communications in Medicine (DICOM) software, which has default settings for conventional CT imaging. The current data on Hounsfield units in CBCT volumes are limited; however, there appears to be some agreement that the grey levels displayed are not representative of Hounsfield units as one would expect from a medical CT scan. However, little has been proposed or published on how to resolve this apparent discrepancy in Hounsfield units between medical CT and CBCT data sets.

In a recent study, a quality control phantom with test inserts of different materials was used to study the relation between CBCT voxel intensity values and medical CT numbers. The phantom included inserts with areas of PMMA, hydroxyapatite in different concentrations, aluminum and air. CT numbers of the different materials were recorded with a medical CT unit and scans of the phantom with a CBCT unit was performed. The correlation between the CBCT voxel intensity values and medical CT numbers was found to be non-linear. Non-uniformity issues were also observed. The use of phantoms was thought to be difficult due to the non-uniformity of measurements and the non-linear relationship observed between the CBCT voxel intensity and the medical CT numbers. This study, along with other similar studies, led to the widespread believe that the use of phantoms in CBCT scanners would not be useful in correlating CBCT pixel intensities with Hounsfield units.

### SUMMARY OF THE INVENTION

In one embodiment, a device for use in a computed tomography system includes a body and one or more radiographic reference objects coupled to, or disposed in or on the body. The body, in some embodiments, is positionable in a mouth of a human subject.

In one embodiment, the device includes at least two radiographic reference objects having different radiodensities. The radiographic reference objects include one or more of: an adipose equivalent material; a water equivalent material; a muscle equivalent material; an inner (cancellous) bone equivalent material; a hard bone (cortical) equivalent material; and a metal.

In one embodiment, a mouthpiece includes a mouthpiece body and a radiographic device coupled to the mouthpiece body. The radiographic device comprises a radiographic device body and one or more radiographic reference objects coupled to, or disposed in or on the body. The body, in some embodiments, is positionable in a mouth of a human subject.

In an embodiment, a method of creating an image of a subject using a computed tomography system, the method includes placing a radiographic device, as described above, proximate to a portion of a human subject. One or more x-ray scans are collected using a computed tomography system, wherein at least a portion of the collected x-ray scans include: (a) at least a portion of the human subject; and (b) at least a portion of the radiographic device positioned proximate to the human subject. An image is then produced from data collected from the x-ray scans. The grey levels of the produced image are correlated to the attenuation coefficients of tissues and/or bones depicted in the produced image, wherein the correlation is based on measured grey levels of one or more radiographic reference objects of the radiographic device.

In one embodiment, correlating grey levels of the produced image to the attenuation coefficients of tissues and/or bones is performed by: selecting a first effective energy of the x-ray beam, based, in part, on the selected peak keV used to collect the scans. A function that correlates the grey levels measured for two or more of the radiographic reference objects to the attenuation coefficients of the two or more radiographic reference objects at the selected effective energy is determined. One or more additional effective energies of the x-ray beam, based, in part, on the selected peak keV used to collect the scans is selected. A function is determined for each selected additional effective energy that correlates the grey levels measured for two or more of the radiographic reference objects to the attenuation coefficients of the two or more radiographic reference objects at the selected additional effective energy. The effective energy is determined by analyzing the accuracy of two or more of the determined functions to correlate the grey levels to the known attenuation coefficients. After analysis of the accuracy of the functions, an accurate function for correlating the grey levels to the known attenuation coefficients is selected and the function is used to determine CT numbers for tissues and/or bones depicted in the produced image.

In another embodiment, correlating grey levels of the produced image to the attenuation coefficient of tissues and/or bones includes selecting an effective energy of the x-ray beam, based, in part, on the selected peak keV used to collect the scans. A function is determined that correlates the grey levels measured for two or more of the radiographic reference objects to the attenuation coefficients of the two or more radiographic reference objects at the selected effective energy. The function is used to determine CT numbers for tissues and/or bones depicted in the produced image.

In an embodiment, a method of creating an image of a subject using a computed tomography system, the method includes placing a radiographic device, as described above, proximate to a portion of a human subject. One or more x-ray scans are collected using a computed tomography system, wherein at least a portion of the collected x-ray scans include: (a) at least a portion of the human subject; and (b) at least a portion of the radiographic device positioned proximate to the human subject. An image is then produced from data collected from the x-ray scans. The grey levels of the produced image are correlated to the CT numbers of tissues and/or bones depicted in the produced image, wherein the correlation is based on measured grey levels of one or more radiographic reference objects of the radiographic device.

In one embodiment, correlating grey levels of the produced image to the CT numbers of tissues and/or bones is performed by: selecting a first effective energy of the x-ray beam, based, in part, on the selected peak keV used to collect the scans. A function that correlates the grey levels measured for two or more of the radiographic reference objects to the CT numbers of the two or more radiographic reference objects at the selected effective energy is determined. One or more additional effective energies of the x-ray beam, based, in part, on the selected peak keV used to collect the scans is selected. A function is determined for each selected additional effective energy that correlates the grey levels measured for two or more of the radiographic reference objects to the CT numbers of the two or more radiographic reference objects at the selected additional effective energy. The effective energy is determined by analyzing the accuracy of two or more of the determined functions to correlate the grey levels to the known CT numbers. After analysis of the accuracy of the functions, an accurate function for correlating the grey levels to the known CT numbers is selected and the function is used to determine CT numbers for tissues and/or bones depicted in the produced image.

In another embodiment, correlating grey levels of the produced image to the CT numbers of tissues and/or bones includes selecting an effective energy of the x-ray beam, based, in part, on the selected peak keV used to collect the scans. A function is determined that correlates the grey levels measured for two or more of the radiographic reference objects to the CT numbers of the two or more radiographic reference objects at the selected effective energy. The function is used to determine CT numbers for tissues and/or bones depicted in the produced image.

In another embodiment, a method of creating an image of a subject using a computed tomography system includes collecting one or more x-ray scans using a computed tomography system, wherein at least a portion of the collected x-ray images scan include: (a) at least a portion of a human subject's teeth; and (b) at least a portion of muscle tissue of a human subject. An image is produced from data collected from the x-ray scans. The grey levels of the produced image are correlated to the attenuation coefficients and/or the CT numbers of tissues and/or bones depicted in the produced image, wherein the correlation is based on measured grey levels of one or more portions of the human subject's teeth and the grey levels of one or more portions of muscle tissue of the human subject.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts an embodiment of a radiographic device;

FIG. 2 depicts an embodiment of a mouthpiece and a radiographic device couplable to the mouthpiece;

FIG. 3 depicts an embodiment of a mouthpiece with a radiographic device coupled to the mouthpiece;

FIGS. 4 and 5 depict examples of the linear fits that were obtained when the grey levels were plotted against the linear attenuation coefficients at a particular effective energy;

FIG. 6 depicts the original distribution of grey levels on the Planmeca ProMax 3D for the two bone equivalent materials in the intra-oral reference object;

FIG. 7 shows the distribution of “corrected” grey levels on the Planmeca ProMax 3D for the two bone equivalent materials in the intra-oral reference object;

FIG. 8 depicts linear regressions of CT number in HU as a function of grey level performed at several arbitrary effective energies; and

FIG. 9 depicts the distribution of “corrected” grey levels on the Planmeca ProMax 3D using the same energy levels.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

In an embodiment, an image of a human subject may be created using a computed tomography (CT) system. In particular, cone beam computed tomography systems (CBCT) or micro computed tomography systems (microCT) may be used to create an image of one or more tissues of a human subject. The image that is created may include one or more indicators that convey information regarding the attenuation coefficients of the features depicted in the image. As used herein the term “attenuation coefficient” refers to the property of relative absorption of x-rays passing through a material. The attenuation coefficients may be determined using a radiographic device positioned in the imaging field of the CT system. The attenuation coefficient of tissues depicted in the image may be determined by relating an optical feature (e.g., grey levels) of the image to the same optical features of the radiographic device, where the radiographic device has one or more radiographic reference objects of known physical composition and properties. The attenuation coefficient of the tissues in the object may be used to produce indicators that depict the CT numbers associated with tissues that appear in the collected image. As used herein the term “radiographic device” refers to any object that includes one or more objects having known composition and physical characteristics that are visible in the x-ray scans collected using a CT system.

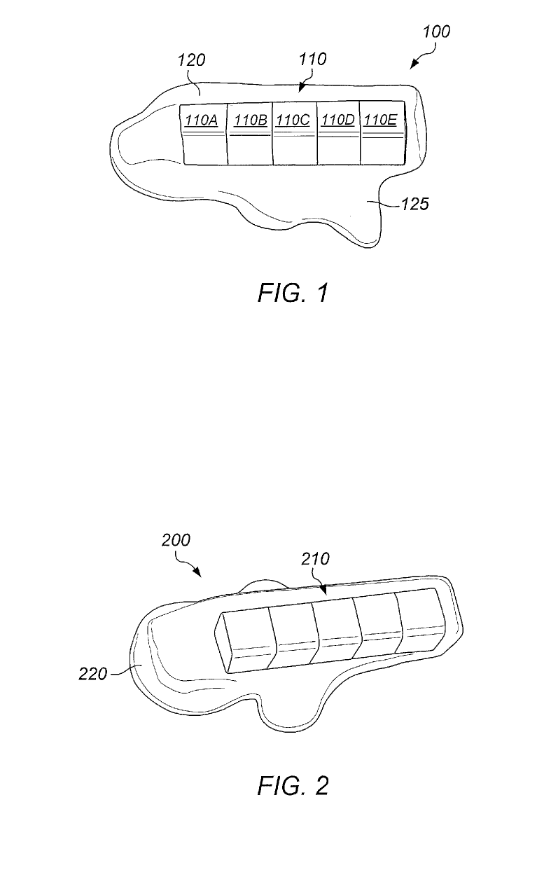

An embodiment of a radiographic device 100 is depicted in FIG. 1. A radiographic device includes one or more radiographic reference objects 110 disposed in a body 120. It should be understood that while the radiographic device depicted in FIG. 1 includes five radiographic reference objects, a radiographic device may include more or less than five radiographic objects. A radiographic device may include at least one radiographic reference object, at least two radiographic reference objects, or at least three radiographic reference objects. Generally, a radiographic device includes at least a sufficient number of radiographic reference objects to provide adequate references for the basis of subsequent calculations.

In some embodiments, each radiographic reference object present in the radiographic device has a different attenuation coefficient. For the determination of bone density using CBCT or micro CT systems, one or more radiographic reference objects for the radiographic device may be chosen to mimic various bone and tissue structures. For example, radiographic reference objects may mimic adipose tissue, muscle tissue, water, inner (cancellous) bone, and hard (cortical) bone. One or more radiographic reference objects that mimic bone and tissue structures may be present in a radiographic device. A radiographic device may also include one or more metal radiographic reference objects or one or more radiographic reference objects formed from a material having a density greater than the density of a hard bone (cortical) equivalent material. Examples of metal radiographic reference objects include aluminum and titanium radiographic reference objects. In some embodiments, a radiographic device includes one or more metal radiographic reference objects and/or one or more radiographic reference objects that mimic bone and tissue structures. Radiographic reference objects may be purchased from Gammex Inc. (Middleton, Wis.). Radiographic reference objects may be natural (e.g., bone samples obtained from a cadaver) or synthetic. Synthetic radiographic reference objects may be formed from a polymeric material that simulates the attenuation coefficient of the mimicked bone or tissue structure. Materials that may be used to simulate bone and tissue structures include, but are not limited to, epoxy resins.

In an embodiment depicted in FIG. 1, a radiographic device includes 5 radiographic reference objects. For example, a radiographic device may include an aluminum reference object 110A, a hard (cortical) bone equivalent reference object 110B, an inner (cancellous) bone equivalent reference object 110C, a polymethyl methacrylate (PMMA) reference object 110D, and a water equivalent reference object 110E. In an embodiment, the radiographic reference objects are positioned next to each other in a line. For ease of identification, the radiographic reference objects may be placed in order of increasing density. For example, the least dense radiographic reference object (e.g., the water equivalent reference object 110E) is placed at one end of the line of radiographic reference objects and the most dense of the radiographic reference objects (e.g., the aluminum reference object 110A) may be placed at the opposite end of the line of radiographic reference objects.

The size and/or shape of the radiographic device may be appropriate for the intended use. For example, when used in oral applications, the radiographic device may have a size that allows for the radiographic device to be inserted in the oral cavity of a subject. For simplicity of manufacturing, one or more of the radiographic reference objects may be in the form of a cube. In some embodiments, the cubic radiographic reference objects have a side length of less than 30 mm, less than 20 mm, less than 10 mm, or less than 5 mm. In some embodiments, the cubic radiographic reference objects have a side length of between about 1 mm to about 30 mm, of between about 1 mm and 20 mm, of between about 1 mm and 10 mm, or of between about 1 mm and 5 mm. In some embodiments, the cubic radiographic reference objects have a side length of greater than 1 mm.

The radiographic device may include one or more reference radiographic objects disposed or coupled to a body 120. Body 120 may be formed from a polymeric resin. Examples of polymeric resins that may be used to form the body include, but are not limited to acrylic polymers (e.g., polymethyl methacrylate) “PMMA”). Other materials may be used. The reference radiographic objects may be embedded in the body. In alternate embodiments, the reference radiographic objects may be coupled to the body. Reference radiographic objects may be removably coupled to the body to allow the reference radiographic objects to be interchanged for different applications.

The radiographic reference objects used in a radiographic device are chosen to have attenuation coefficients that can be used to create a correlation function that relates an optical characteristic of an image to the attenuation coefficient of the feature depicted in the image. For example, grey levels of an image may be correlated to attenuation coefficients based on grey levels measured for one or more of the radiographic objects of the radiographic device.

In one embodiment, the body 120 of the radiographic device may be formed in the shape of a mouthpiece. For example, body 120 may include one or more indentations 125 that may correspond to the profile of the oral cavity of a human subject. The radiographic device, in such embodiments, has a size that allows the device to be positioned inside the oral cavity of a human subject. In some embodiments, the radiographic device may be formed from a sterilizable material. After use, the radiographic device may be sterilized using standard sterilization techniques (e.g., gas, plasma, chemical, or thermal techniques (e.g., steam treatment or hot air treatment) to allow use with other patients.

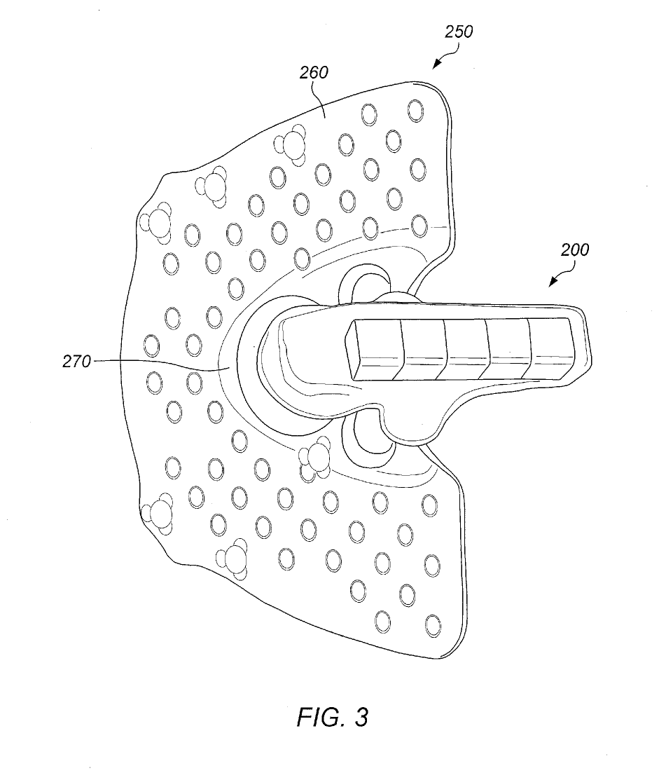

In another embodiment, the radiographic device may be removably coupled to a mouthpiece. An embodiment of a radiographic device that may be coupled to a mouthpiece is depicted in FIG. 2. In FIG. 2, a radiographic device 200 includes one or more radiographic reference objects 210. For example, a radiographic device 200 may include 5 radiographic reference objects, as described in FIG. 1.

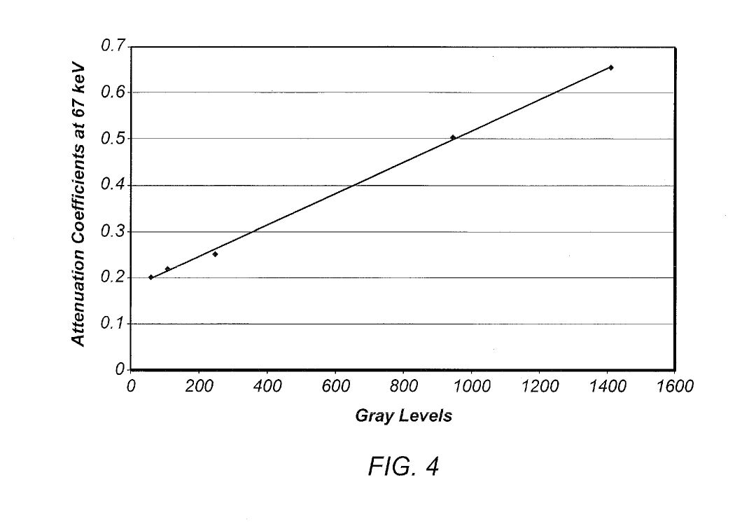

A mouthpiece 250 that may be coupled to a radiographic device is depicted in FIG. 3. In one embodiment, radiographic device 200 may be couplable to a mouthpiece 250, as shown in FIG. 3B. Mouthpiece 250 may include a mouthpiece body 260. In one embodiment, device 200 is couplable to body 260 of mouthpiece 250. During use, mouthpiece 250, with radiographic device 200 coupled to body 260, is positionable in a mouth of a human. In one embodiment, radiographic device 200 is coupled to mouthpiece 250 such that the radiographic device is disposed inside the human subject's mouth, when the mouthpiece is placed in a human mouth. When the mouthpiece is disposed inside of the subject's mouth, the radiographic device, in some embodiments, may be positioned above the occlusion area of the teeth. In an alternate embodiment, not shown, the radiographic device may be positioned outside of the mouth when the mouthpiece is positioned in the mouth.

In one embodiment, radiographic device 200 is removably coupleable to mouthpiece body 260. In one embodiment, radiographic device 200 includes a coupling element 220 which mates with a complementary element 270 of mouthpiece 250. For example, coupling element 220 may be a rounded projection, as shown in FIG. 2. Coupling element 270 may be an opening which has a size a shape that is capable of receiving rounded coupling element 220.

Body 260 of mouthpiece 250 may be a commercially available mouthpiece. For example, in one embodiment, mouthpiece 250 may be a commercial biteplate that is used for x-ray imaging (e.g., a Sirona acrylic biteplate commercially available from Sirona Dental Systems LLC, Charlotte N.C.). When a commercially available mouthpiece is used, radiographic device 200 may be configured to couple to an existing coupling element of the commercially available mouthpiece.

In one embodiment, an image of a subject may be created using a CT system (e.g., a CBCT system or a microCT system) and a radiographic device. In an embodiment, a radiographic device is placed proximate to a portion of a human subject that is going to be imaged. In an embodiment in which the CT system will be imaging the mouth of a subject, the radiographic device may be placed in the mouth of the subject. Alternatively, the radiographic device may be placed on the skin of the subject, proximate to the portion of the subject being imaged. For example, if the back of the subject is being imaged, a radiographic device may be placed on the skin proximate to the portion of the back that is being imaged.

After the radiographic device is positioned on, or in, the subject, one or more x-ray scans of at least a portion of the subject and at least a portion of the radiographic device are collected. The scans collected may be saved using the CT system software and exported as a data file suitable for analysis. For example, the collected scans may be exported using the Digital Imaging and Communications in Medicine (“DICOM”) standard. This allows the data to be analyzed using third party or proprietary software.

Data from the collected scans is used to produce an image of the scanned region of the subject. Any third party software, or proprietary imaging software, having the capability of analyzing the grey levels of the produced image may be used. In one embodiment, 3D viewing software is used when a composite image is analyzed. 3D viewing software that may be used includes, but is not limited to, On Demand 3D® software (CyberMed International, Seoul, Korea). Analysis of the produced images was performed by obtaining the mean grey level in a region of interest in the produced images using the imaging software. The mean grey levels of the radiographic device were also collected using the imaging software.

After collection of the grey level data, a correlation was made relating grey levels of the produced image to the attenuation coefficients of the tissues and/or bones depicted in the image. The correlation is derived, in part, based on the grey level data of the radiographic reference objects of the radiographic device. In one embodiment, a function relating the measured grey levels of the radiographic device to attenuation coefficients is derived.

In one embodiment, the correlation relating grey levels to attenuation coefficients may be derived by collecting data from a scan taken of at least a portion of a human subject, and at least a portion of a radiographic device positioned proximate to the human subject. The collected data may be used to form an image that includes grey levels for the tissues and bones as well as grey levels of one or more of the radiographic reference objects of the radiographic device.

CT systems use x-ray devices that output x-rays that are polychromatic or heterogeneous in energy, i.e., the x-ray beams contain a spectrum of photon energies. Thus, although a maximum energy value is selected when the CT system is operated, the actual energy values of the x-ray photons exhibit a spectrum of energies. Generally, the energy level selected for use in a CT system is the peak kV (or kVp). The peak kV determines the maximum photon energy of x-rays produced by the device during the scan. Although a spectrum of x-ray energy is produced, the energy of the x-ray photons may be characterized by the effective energy value. As used herein the “effective energy” of an x-ray beam is an energy that gives the same attenuation results as the spectrum of photons actually used to generate the CT scans. For example, a CBCT scanner operating at 80 kVp exhibits an effective energy of 63 keV.

The “attenuation coefficient” of a material describes the extent to which the intensity of an energy beam (e.g., an x-ray beam) is reduced as it passes through a specific material. The attenuation coefficient of a material varies depending on the type of energy beam passing through the material and the energy of the energy beam. In a CT scan, as the energy of the x-ray photons is altered, the attenuation coefficient of the material changes. Table 1 depicts a sample of attenuation coefficients for various materials over a range of 10 keV.

TABLE 1

| keV | Aluminum | Hard Bone | Inner Bone | Water |
|---|---|---|---|---|
| 70 | 0.621 | 0.47672983 | 0.240998005 | 0.192665720 |
| 71 | 0.6129 | 0.469041785 | 0.239033653 | 0.191665720 |
| 72 | 0.6021 | 0.461794410 | 0.236689644 | 0.190777618 |
| 73 | 0.594 | 0.454483950 | 0.234269130 | 0.189777618 |
| 74 | 0.5859 | 0.448250560 | 0.232625479 | 0.188777618 |
| 75 | 0.5778 | 0.441436825 | 0.230307851 | 0.187777618 |
| 76 | 0.5724 | 0.435153115 | 0.228151376 | 0.186777618 |
| 77 | 0.5643 | 0.429416450 | 0.226610611 | 0.185777618 |
| 78 | 0.5562 | 0.423658510 | 0.224611046 | 0.184889516 |
| 79 | 0.5508 | 0.419127860 | 0.223502470 | 0.184777618 |

The attenuation coefficient on each of the radiographic reference objects is known or derived. If the radiographic reference objects are purchased, the attenuation coefficients, at various effective energies, may be available from the supplier. If the attenuation coefficients are not know, the attenuations coefficients may be derived using the National Institute of Standards and Technology (NIST) tables of x-ray mass attenuation coefficients and mass energy absorption coefficients. The data supplied by NIST are tabulated from 1 keV to 20 keV. Since most CBCT systems operate in the range of 30 keV to 150 keV, it may be necessary to interpolate the NIST data for this range. To determine the attenuation coefficients of a radiographic reference object, the elemental composition of the radiographic reference object is determined or obtained from the manufacturer. Elemental composition may be determined for a sample using known techniques (e.g., combustion analysis). Using the elemental composition, and the attenuation coefficients for each element forming the radiographic reference object, the attenuation coefficients for the object can be determined. In some embodiments, attenuation coefficients for a radiographic reference object are determined at different energy levels. For example, attenuation coefficients may be determined at energy levels from 30 keV to 150 keV in 1 keV increments.

If the effective energy of a CT scanner is known for a CT scan, the grey levels of objects appearing in produced images may be correlated to attenuation coefficients by looking up the known attenuation coefficients at the effective energy. It is, however, difficult, if not impossible, to measure or even determine the effective energy of the x-ray photons for any given scan. Thus, the determination of the attenuation coefficients of tissues and bones in a CT scan from the grey levels measured has been difficult.

The effective energy of a CT scan may be determined by correlating the grey levels of one or more of the radiographic reference objects of a radiographic device with the known attenuation coefficients associated with the radiographic reference objects. In an embodiment, x-ray scans of a portion of a human subject and at least a portion of a radiographic device positioned proximate to the human subject are collected. The radiographic device includes one or more radiographic reference objects having known attenuation coefficients. In order to determine the effective energy of the x-ray photons used to collect the scans, a first effective energy of the x-ray beam is arbitrarily selected. In some embodiments, the selected first effective energy will be about 10% to about 80% of the peak effective energy, about 20% to about 60% of the peak effective energy, or about 30% to about 40% of the peak effective energy. A function that correlates the grey levels measured for two or more of the radiographic reference objects to the known attenuation coefficients of the radiographic reference objects at the selected first effective energy is determined.

Generally, the function correlating grey levels to attenuation coefficients is a linear function generated by plotting the grey values of two or more of the radiographic reference objects to the known attenuation coefficients of the two or more radiographic reference objects at the selected first effective energy. This process may be repeated using different effective energies by generating a new plot using the attenuation coefficients that are associated with the selected effective energy. In some embodiments, a range of effective energies are analyzed in this manner generating a plurality of linear functions which correlate the grey levels to the attenuation coefficient at each of the selected effective energies. In some embodiments the grey levels for each radiographic reference object was plotted against attenuation coefficients at 1 keV increments in a range of about 10, keV, of about 20 keV, of about 30 keV, of about 40 keV, of about 50 keV, of about 6t0 keV of about 70 keV, of about 80 keV of about 90 keV, or of about 100 keV.

After generating a plurality of linear plots of grey levels against attenuation coefficient, a linear regression may be used to analyze the accuracy of each plot with respect to the data at each selected effective energy. In one embodiment, a linear regression was performed for each effective energy in the relevant range until accurate linear fits were obtained. The R² value is determined for each plot and the plot or plots having the best accuracy are used. Generally, a plot at an accurate selected effective energy will exhibit an R² value of at least 0.99, at least 0.999, at least 0.9999, or at least 0.99995. Generally it was seen that, with increasing effective energy, the R² value climbs to a maximum at some particular energy and then decreases as the effective energy continues to rise. In some embodiments, the accurate plot, and thus the accurate function for correlating the grey levels to attenuation coefficients is selected as the function whose plot exhibits the greatest R² value.

Once the accurate function has been determined, the attenuation coefficient of any feature depicted in the produced image can be determined using the linear function.

The attenuation coefficient of a bone or tissue in a body is proportional to the CT number for the bone or tissue. Specifically, the CT number is calculated using the equation:

CT=(μₘₐₜₑᵣᵢₐₗ−μ_{water})/(μ_{water})×k

Where μₘₐₜₑᵣᵢₐₗ is the attenuation coefficient of the studied feature and μ_{water} is the attenuation coefficient of water; and k is a constant. Thus, using the linear function derived from the reference radiographic objects, the attenuation coefficient, and thus the CT number of any feature depicted in a produced image can be determined.

Hounsfield units are a commonly used unit for describing the CT number of a feature depicted in a CT scan. In some embodiments, Hounsfield units may be determined for one or more features depicted in the produced image. Once the attenuation coefficients have been determined for the features depicted in a produced image, the Hounsfield units for any of the depicted features may be derived from the formula:

HU=(μₘₐₜₑᵣᵢₐₗ−μ_{water})/(μ_{water})×1000

Thus, Hounsfield units are CT numbers calculated using k=1000. In this manner, grey levels of an image produced using a CBCT system may be used to calculate Hounsfield units. By deriving Hounsfield units from grey levels, the images produced from different CT systems or from the same CT system collected at different times, may be directly compared.

While determining the effective energy of a CT scan was shown to give an accurate correlation of the grey levels to the attenuation coefficients, it was surprisingly found that a detailed statistical analysis of multiple possible effective energies was not needed. In an alternate embodiment, the correlation relating grey levels to attenuation coefficients may be derived by collecting data from a scan taken of at least a portion of a human subject, and at least a portion of a radiographic device positioned proximate to the human subject. The collected data includes grey levels for the tissues and bones depicted in one or more of the images produced from the collected data. The produced images also include grey levels of one or more of the radiographic reference objects of the radiographic device. In an embodiment, an arbitrary effective energy of is selected. In some embodiments, the arbitrary effective energy will be selected in a range of about 10% to about 80% of the peak effective energy, about 20% to about 60% of the peak effective energy, or about 30% to about 40% of the peak effective energy. A function that correlates the grey levels measured for two or more of the radiographic reference objects to the known attenuation coefficients of the radiographic reference objects at the arbitrary selected effective energy is determined.

In a surprising discovery, it has been found that at most arbitrarily selected energies, the function correlating grey levels to attenuation coefficients is sufficiently accurate to allow determination of the attenuation coefficients, and subsequently, CT numbers and Hounsfield units. Thus, after the linear function correlating grey levels to Hounsfield units at the selected energy is determined, CT numbers and/or Hounsfield units for tissues and/or bones that appear in the CT scans may be determined using the determined linear function, as described above.

In alternate embodiments, CT numbers or Hounsfield units may be plotted directly with respect to grey levels to allow the direct determination of CT number or Hounsfield units of bone or tissues depicted in the produced images, without having to determine the attenuation coefficient of these features. For example, in a method, the effective energy of a CT scan may be determined by correlating the grey levels of one or more of the radiographic reference objects of a radiographic device with the calculated CT numbers or Hounsfield units associated with the radiographic reference objects. In an embodiment, x-ray scans of a portion of a human subject and at least a portion of a radiographic device positioned proximate to the human subject are collected. The radiographic device includes one or more radiographic reference objects having known attenuation coefficients. In order to determine the effective energy of the x-ray photons used to collect the scans, a first effective energy of the x-ray beam is arbitrarily selected. In some embodiments, the selected first effective energy will be about 10% to about 80% of the peak effective energy, about 20% to about 60% of the peak effective energy, or about 30% to about 40% of the peak effective energy. A function that correlates the grey levels measured for two or more of the radiographic reference objects to the calculated CT numbers or Hounsfield units of the radiographic reference objects at the selected first effective energy is determined. The calculation of the CT numbers or Hounsfield numbers at the selected first effective energy is readily accomplished for the radiographic reference objects since the attenuation coefficients for the radiographic reference objects is known.

Generally, the function correlating grey levels to CT numbers is a linear function generated by plotting the grey values of two or more of the radiographic reference objects to the calculated CT numbers of the two or more radiographic reference objects at the selected first effective energy. This process may be repeated using different effective energies by generating a new plot using calculated CT numbers that are associated with the selected effective energy. In some embodiments, a range of effective energies are analyzed in this manner generating a plurality of linear functions which correlate the grey levels to the CT numbers at each of the selected effective energies. In some embodiments the grey levels for each radiographic reference object was plotted against calculated CT numbers at 1 keV increments in a range of about 10, keV, of about 20 keV, of about 30 keV, of about 40 keV, of about 50 keV, of about 6t0 keV of about 70 keV, of about 80 keV of about 90 keV, or of about 100 keV.

After generating a plurality of linear plots of grey levels against calculated CT numbers, a linear regression may be used to analyze the accuracy of each plot with respect to the data at each selected effective energy. In one embodiment, a linear regression was performed for each effective energy in the relevant range until accurate linear fits were obtained. The R² value is determined for each plot and the plot or plots having the best accuracy are used. Generally, a plot at an accurate selected effective energy will exhibit an R² value of at least 0.99, at least 0.999, at least 0.9999, or at least 0.99995. Generally it was seen that, with increasing effective energy, the R² value climbs to a maximum at some particular energy and then decreases as the effective energy continues to rise. In some embodiments, the accurate plot, and thus the accurate function for correlating the grey levels to CT numbers is selected as the function whose plot exhibits the greatest R² value.

Once the accurate function has been determined, the CT numbers of any tissue and/or bone features depicted in one or more of images produced from the collected data may be determined using the selected linear function.

While determining the effective energy of a CT scan was shown to give an accurate correlation of the grey levels to the CT numbers, it was surprisingly found that a detailed statistical analysis of multiple possible effective energies was not needed. In an alternate embodiment, the correlation relating grey levels to CT numbers may be derived by collecting data from a scan taken of at least a portion of a human subject, and at least a portion of a radiographic device positioned proximate to the human subject. The images produced using the collected data include grey levels for the tissues and bones and grey levels of one or more of the radiographic reference objects of the radiographic device. In an embodiment, an arbitrary effective energy of is selected. In some embodiments, the arbitrary effective energy will be selected in a range of about 10% to about 80% of the peak effective energy, about 20% to about 60% of the peak effective energy, or about 30% to about 40% of the peak effective energy. A function that correlates the grey levels measured for two or more of the radiographic reference objects to the calculated CT number of the radiographic reference objects at the arbitrary selected effective energy is determined After the linear function correlating grey levels to CT numbers at the selected energy is determined, CT numbers and/or Hounsfield units for tissues and/or bones that appear in the CT scans may be determined using the determined linear function directly.

CBCT imaging is rapidly becoming a standard of care in dentistry and is perceived as a benefit for the patient and clinician in terms of improved treatment outcome. While a CBCT scan is also useful to determine bone quantity, bone height and proximity of adjacent structures, it lacks the ability to quantify bone quality in a meaningful manner. Applicant's disclosed methods may be used to derive Hounsfield units using grey levels in the CBCT volume. Application of this method to derive Hounsfield units can provide a standardized method to assess bone quality similar to that found in medical CT.

Since dental implant technology and the concept of osseointegration has evolved, the emphasis on bone morphology and quality have received great importance in the prediction of implant success. Several studies have attempted to classify bone quality both pre-operatively and during implant placement. Pre-operative implant site assessment is the preferred method; however, it has somewhat limited success in prediction of implant success.

When 3D imaging became available with medical CT scanners several studies attempted to classify bone quality using bone density values. One such study was done using a spiral CT (GE ProSpeed helical scanner; General Electric, Slough, UK). This study found a range of bone densities for dental implants: anterior mandible >850 HU; posterior mandible/anterior maxilla +500 to 850 HU; posterior maxilla 0 to 500 HU; tuberosity area <0 HU). Another pre-operative radiological study was done on patients using a spiral CT machine (Siemens AR-SP 40, Munich, Germany) for assessing bone volume and to determine the correlation between bone density, insertion torque and dental implant stability. The average radiographic bone density was measured and the average Hounsfield units values based on 4 different regions in the mouth ranged from 459 HU to 928 HU. The highest Hounsfield units mean is from the anterior mandible and the lowest Hounsfield units mean is from the posterior maxilla. In another retrospective dental clinical study implant recipient sites were evaluated with a spiral CT machine (Siemens Somatom AR-SP 40, Erlanger, Germany). The mean bone density of the implant recipient area was measured again in Hounsfield units using a CT machine software.

Carl E. Misch in 1999 developed a bone classification scale: D1 bone >1250 HU; D2 bone 850-1250 HU; D3 bone 350-850 HU; D4 bone 150-350 HU; D5 bone <150 HU. D1 is described as dense cortical bone and found only in the mandible about 8% of the time. D2 is described as having a dense cortex with a course trabecular bone pattern and this is the most common bone density in the mandible. D3 is described as having a thin cortex and a fine trabecular bone pattern and this is the most common bone density found in the maxilla. D4 is fine trabecular bone found primarily in the posterior maxilla. D5 is very soft bone and is usually consistent with sinus graft augmentation.

Applicant's described methods to derive Hounsfield units using grey levels in CBCT allows the clinician to make meaningful decisions in implant planning, diagnosis, surgical interventions, treatment planning and 2D and 3D reconstruction of images. For example, a clinician will have more opportunity to select alternative implant sites or the orthodontist using mini-implants for anchorage more choices in anchorage sites for treatment. Pre-operative planning would allow the clinician to evaluate the bone density in the CBCT scan based on the Hounsfield units prior to the surgery procedure and plan accordingly on the type of implant to be utilized, length and shape of implant as well as whether to submerge or not, immediate loading versus osseous integration and healing period prior to loading, and a host of other treatment variables as well as make clinical treatment time more productive. This will result in better patient outcomes and acceptance in implant dentistry, cosmetic reconstruction, complex surgical procedures and other dental treatments.

In order to help a dental professional make informed decisions regarding operative procedures, a CT system may produce an image that includes one or more indicators of the relative density of one or more tissues depicted in the image. The indicators of the densities of one or more of the tissues may be determined using the correlation derived from data collected for the radiographic device. In one embodiment, an indicator of the density of one or more tissues may be displayed as a number that appears in the vicinity of the tissue (e.g., a number representing Hounsfield units). In some embodiments, the indicator is displayed upon receiving a command form the user. For example, moving a mouse over the area may cause the indicator of density to be displayed in the area corresponding to the location of the mouse cursor. In other embodiments, one or more indicators of the density of one or more tissues may include colored regions. For example, regions of a displayed image may be colored to correspond to the Misch bone classification scale. Different colors may be used, for example, for bone regions having a density that corresponds to the density range set forth for D1-D5. Other indicators of density may be used.

Currently, CBCT manufacturers have provided grey levels which are not actual Hounsfield units making it difficult to assess bone quality from a CBCT data set. A method that converts grey levels taken from CBCT data sets into Hounsfield units would standardize and allow comparison of bone quality from machine to machine within a small range.

In some embodiments, a radiographic device is used during CT scanning of each human subject and the correlation is derived for each imaging session. While this method ensures accurate density results, it may not be practical or possible to always perform a CT scan that includes both a radiographic device and a region of interest. In an alternate embodiment, a CT system may be calibrated using a radiographic device. After the radiographic device is positioned on, or in, the subject, one or more x-ray scans of at least a portion of the subject and at least a portion of the radiographic device are collected. A correlation of grey levels of one or more of the images produced from the collected scans to the attenuation coefficients of tissues depicted in one or more of the produced images is created. The correlation is based on measured grey levels of one or more radiographic reference objects of the radiographic device. This correlation may be applied to subsequent CT scans that are performed in the absence of a radiographic device to determine CT numbers for tissue and/or bone depicted in the obtained CT scan.

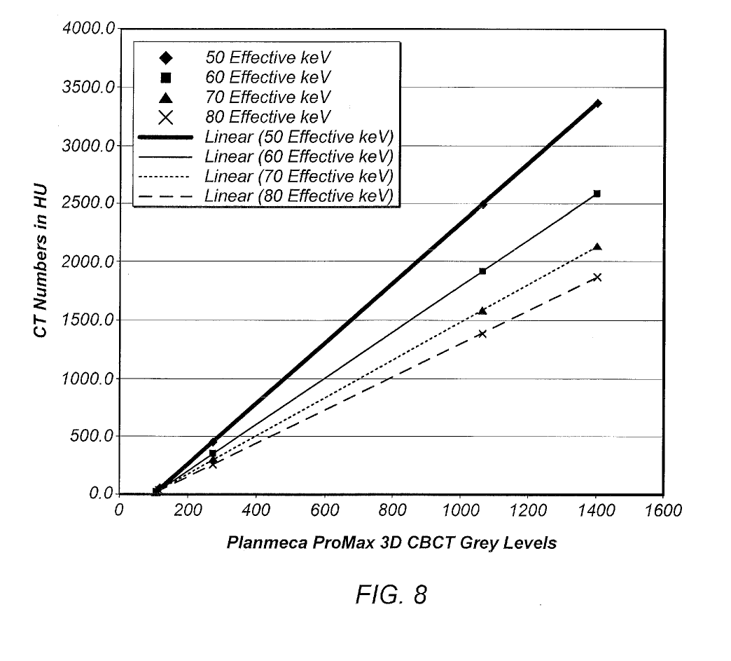

Experimental testing of various CT systems has shown that the relationship between grey levels and attenuation coefficient of the imaged region varies based on the energy level used to collect the image data. For example, FIG. 8 shows the correlation between grey levels and Hounsfield units values at different energy levels for a CBCT system. As can be seen, as the energy level is altered the correlation changes. It should be noted, however, that at any given energy level the correlation is linear. This property of the correlations between grey level and Hounsfield units allows a user to calibrate a CT system such that a radiographic device may not be needed for each imaging session, regardless of the effective energy used.

Operation of a CT system may be performed automatically using an internal controller coupled to various components of the CBCT system, including an x-ray device and a display device coupled to the controller. A controller includes one or more processors and internal memory. Methods used to operate and monitor a CBCT system may be implemented by program instructions stored in memory or a carrier medium coupled to the controller, and executed by one or more processors. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Rambus Random Access Memory (RAM), etc.; or a non-volatile memory such as a magnetic media, e.g., a hard drive, or optical storage. The memory medium may comprise other types of memory as well, or combinations thereof. In addition, the memory medium may be located in a first computer in which the programs are executed, or may be located in a second different computer that connects to the first computer over a network, such as the Internet. In the latter instance, the second computer may provide program instructions to the first computer for execution. The term “memory medium” may include two or more memory mediums that may reside in different locations, e.g., in different computers that are connected over a network.

In some embodiments, a controller includes a processor that includes, for example, one or more field programmable gate arrays (FPGAs), microcontrollers, etc. included on a circuit board disposed in CBCT system. The processor is capable of executing programming instructions stored in a memory of the controller. In some embodiments, programming instructions may be built into the processor such that a memory external to the processor may not be separately accessed (i.e., the memory may be internal to the processor).

Processor may be coupled to a display device. The display device may display an image created during a CBCT system scan. In an embodiment, an image that includes one or more indicators of the Hounsfield units of one or more bones or tissues depicted in the image may be produced by the processor and displayed on the display device. In an embodiment, an indicator of the Hounsfield units of one or more bones of tissues depicted in the image may be a displayed. Alternatively, different colors may be displayed on the image to indicate different Hounsfield unit ranges of the depicted features. The indicators of Hounsfield units may be displayed continually or when requested (e.g., when a mouse cursor passes over a feature).

In an alternate method, an image of a subject may be formed using a CT system. The method includes collecting one or more x-ray scans using a computed tomography system, wherein at least a portion of the collected x-ray images comprise: (a) at least a portion of a human subject's teeth; and (b) at least a portion of muscle tissue of a human subject. The density and related properties of teeth (e.g., Hounsfield units and attenuation coefficients) of the various components of teeth are well known and generally similar between different subjects. Teeth include a plurality of layers having different densities. For example, teeth include enamel, dentin, cementum, and pulp. Each of these layers has a different, but known attenuation coefficient. The attenuation coefficient of muscle tissue is generally well known as well. In dental applications, the tongue is generally visible in an image of teeth. Using the various tooth layers and muscle tissues visible in a CT scan, a correlation between grey levels and the attenuation coefficients or CT numbers of the tissues may be derived.

In one embodiment, grey levels of one or more of the collected images may be correlated to the attenuation coefficients or the CT numbers of tissues depicted in one or more of the collected images. The correlation is based on measured grey levels of one or more portions of the human subject's teeth and the grey levels of one or more portions of muscle tissue of the human subject. As described above, the correlation may be used to produce an image that includes one or more indicators of relative density.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The effectiveness of deriving HU using grey levels in two CBCT systems was investigated in a clinical study

### Patient Selection

The patients selected to participate in this clinical study were prescribed a CBCT by their dental care provider. The patients were asked to participate in the study. Their panoramic radiographs were pre-screened by the primary investigator for pre-existing metallic restorations, endodontic obturation materials, metallic crowns and fixed partial dentures that might create severe artifacts. In order to achieve a realistic perspective, the patients selected for this study were allowed to participate if they had few to multiple metallic restorations while some patients were completely edentulous. Any patient that had full mouth reconstruction or was undergoing active orthodontics and those patient's that were prescribed CBCT scans for pathology, trauma or TMJ disorders were excluded as the placement of the biteplate may alter the diagnostic value of the CBCT scan. Patient radiation dose was not increased as a result of participating in the study. The clinical study was approved by the institutional review board on human subjects under IRB Protocol #HSC20090033H.

### Intra-Oral Reference Object

An intra-oral reference object that is composed of 5 materials was used in the study. The reference materials used were: aluminum, hard (cortical) bone equivalent material, inner (cancellous) bone equivalent material, polymethyl methacrylate (PMMA), and water equivalent material. All materials used were epoxy resin based tissue radiologic substitutes from Gammex RMI Middleton, Wis., except for the aluminum material (1100 aluminum alloy) and PMMA. Each material was cut to the size of a 5×5×5 mm cube and embedded in clear dental acrylic. A total of 3 intraoral reference objects were constructed. The reference object was designed with a smooth finish and rounded corners on the exterior to aid in patient comfort. (FIG. 3)

The intraoral reference object has an acrylic key on the inferior aspect in order to lock onto the top of an acrylic bite plate made by Sirona Dental (Bensheim, Germany). The acrylic bite plate was used to ensure that the intraoral reference object was consistently positioned in the palatal vault of the patient's mouth. The patient was instructed to gently bite down on acrylic bite plate prior to the scan. If the patient was edentulous and had complete dentures, they were left in the mouth to help stabilize the device. For infection control the intraoral device and acrylic bite plate were sterilized using ethylene oxide gas.

### Image Acquisition

Two different CBCT machines were used: the Planmeca ProMax 3D (Planmeca Helsinki, Finland) and the Asahi Alphard 3030 (Belmont Takara, Kyoto, Japan). Thirty adult patients were scanned with each CBCT device using the manufacturer suggested clinical settings for kVp and mA.

In the case of the Asahi Alphard 3030, the scanner rotates 360 degrees around the patient's head resulting in 510 projections. Twenty-five scans were acquired in I-mode with an imaging volume of 102×102 mm and a voxel size of 0.2 mm. Four scans were acquired in P-mode with an imaging volume of 154 mm×154 mm and a voxel size of 0.3 mm. One scan was acquired in C-mode which is a 200×178 mm imaging volume and at a voxel size of 0.39 mm. The scanning parameters were fixed at 80 kVp, 5 mA and 17 seconds for all patients. The Asahi Alphard 3030 has a 30×30 cm flat panel amorphous silicon detector and a 14 bit grey scale for density readings. The equipment specifications for the Asahi Alphard 3030 are summarized in Table 2 below.

TABLE 2
Asahi Alphard 3030 specifications and scan details

| | Number | | |
|---|---|---|---|
| Exposure | of Scans | Exposure area | Voxel size |
| mode | acquired | (mm) | (mm) |
| C-mode | 1 | 200 × 178 (H) | 0.39 |
| P-mode | 4 | 154 × 154 (H) | 0.3 |
| I-mode | 25 | 102 × 102 (H) | 0.2 |
| D-mode | 0 | 51 × 51 (H) | 0.1 |

| | |
|---|---|
| Number of projections | 510 |
| Exposure factors | 80 kV, 5 mA, 17 seconds |
| Degrees of rotation | 360 |
| Flat Panel detector size | 30 × 30 cm |

On the Planmeca ProMax 3D, the scanner rotates only 194 degrees around the patient's head resulting in 300 projections. Twenty-six of the scans were acquired in 80×80 mm volumes in a normal resolution setting with a voxel size of 0.32 mm. Four scans were acquired in 80×80 mm in a high resolution setting with a voxel size of 0.16 mm. The scanning parameters ranged from 80 kVp and 8 mA to 84 kVp and 14 mA for 18 seconds. The Planmeca ProMax 3D has a 12.16×12.16 cm flat panel amorphous silicon detector and uses a 12 bit grey scale for data processing. The equipment specifications for the Planmeca ProMax 3D are summarized in Table 3 below. Since the time of this study, Planmeca has upgraded the acquisition and reconstruction software on their proprietary Romexis software from a 12 to 15 bit grey level.

TABLE 3
Planmeca ProMax 3D specifications and scan details

| | Number | | |
|---|---|---|---|
| Exposure | of Scans | Exposure area | Voxel size |
| mode | acquired | (mm) | (mm) |
| Adult | 26 | 80 × 80 (H) | 0.32 |
| Normal resolution | | | |
| Adult | 4 | 80 × 80 (H) | 0.16 |
| Enhanced resolution | | | |

| | |
|---|---|
| Number of projections | 300 |
| Exposure factors | 80 kV, 8 mA, 18 seconds |
| | 82 kV, 10 mA, 18 seconds |
| | 84 kV, 12 mA, 18 seconds |
| | 84 kV, 14 mA, 18 seconds |
| | 84 kV, 16 mA, 18 seconds |
| Degrees of rotation | 194 |
| Flat Panel detector size | 12.16 × 12.16 cm |

The raw images were processed and reconstructed using the manufacturer's proprietary software. The images were then exported in DICOM format for data analysis.

While there were 30 patients scanned on each CBCT scanner, the data was averaged for only 29 patients in each case. With the Asahi Alphard scanner, one patient was removed from the study because endodontic filling material caused significant streak artifacts through the area of the reference object. In the case of the Planmeca ProMax 3D scanner, one patient swallowed or respirated during scan resulting in a vertical streak artifact in the middle of the scan impairing visualization of the reference object.

### Image Evaluation

The DICOM data sets were imported into a third party viewing software, On Demand 3D® (Cybermed, Seoul, Korea) to determine the grey levels in each of the materials for each scan. The slice thickness was not increased and was dependent upon the voxel size of each scan.

Grey level samples were taken from a square 10×10 pixel region of interest (ROI) for all 5 materials from both CBCT machines. Any sagittal, coronal or axial view could have been used, but primarily the sagittal views were used for sampling. The ROI was moved around anywhere within the 5×5×5 mm block of the reference material. The highest attainable grey levels in the ROI were chosen for the 1100 aluminum alloy, outer bone equivalent material (cortical bone) and inner bone equivalent material (trabecular bone). For consistency the linear attenuation coefficients followed a specific order from high to low: aluminum>outer bone equivalent>inner bone equivalent>PMMA>water.

The grey levels for each material were analyzed using a regression analysis with the calculated attenuation coefficients for each material at 1 keV increments. The best fit or regression value of one was selected as the effective energy of the CBCT scan.

Theoretical CT numbers in HU were determined from the linear attenuation coefficients at the above determined effective energy of each of the 5 reference materials using the standard equation:

HU=(μₘₐₜₑᵣᵢₐₗ−μ_{water})/(μ_{water})×1000

These CT numbers were compared to the CT numbers calculated using the grey levels of each of the materials within the region of interest using the regression equation and the difference was expressed as a percentage of the range of the HU scale of the materials in the reference object.

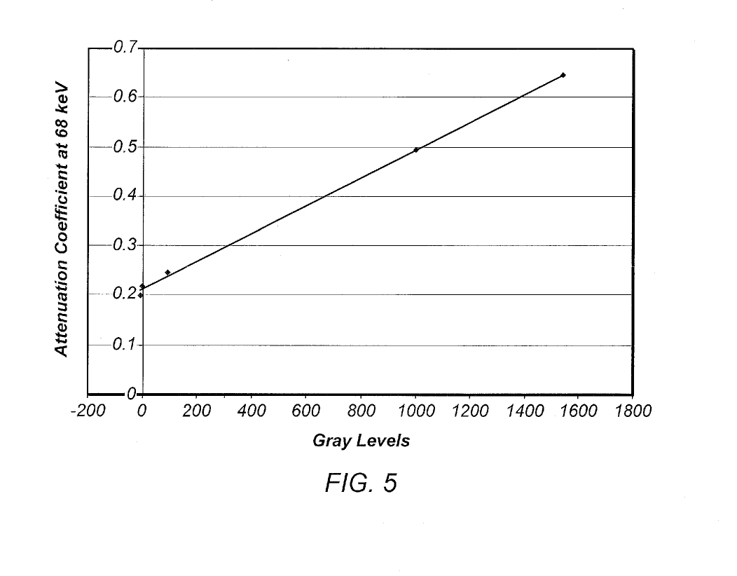

FIGS. 4 and 5 are examples of the linear fits that were obtained when the grey levels were plotted against the linear attenuation coefficients at a particular effective energy. The water equivalent material has the lowest value and the aluminum has the highest value for both CBCT scanners.

Tables 4 and 5 show the mean values for effective energy, grey level, calculated HU, actual HU and percent difference. The overall variability between the actual and calculated HU was less than three percent.

TABLE 4
Average Values for Asahi Alphard 3030

| | Original | Calculated | Actual | Percentage |
|---|---|---|---|---|
| Mean keV 65.9 | Grey Level | HU | HU | Difference |
| Aluminum | 1428.5 | 2503.4 | 2484.8 | 0.80 |
| Outer Bone | 939.7 | 1671.1 | 1695.2 | 1.50 |
| Equivalent | | | | |
| Inner Bone | 157.3 | 302.4 | 296.0 | 1.83 |
| Equivalent | | | | |
| PMMA | 46.0 | 90.3 | 103.2 | 1.15 |
| Water | 22.4 | 47.0 | 19.4 | 1.93 |
| Equivalent | | | | |

TABLE 5
Average Values for ProMax 3D

| | Original | Calculated | Actual | Percentage |
|---|---|---|---|---|
| Mean keV 74.9 | Grey Level | HU | HU | Difference |
| Aluminum | 1384.5 | 2191.4 | 2177.6 | 0.99 |
| Outer Bone | 823.9 | 1401.3 | 1419.4 | 2.09 |
| Equivalent | | | | |
| Inner Bone | 23.8 | 249.5 | 241.7 | 2.01 |
| Equivalent | | | | |
| PMMA | −81.0 | 86.5 | 116.5 | 1.56 |
| Water | −101.1 | 56.0 | 16.0 | 2.68 |
| Equivalent | | | | |

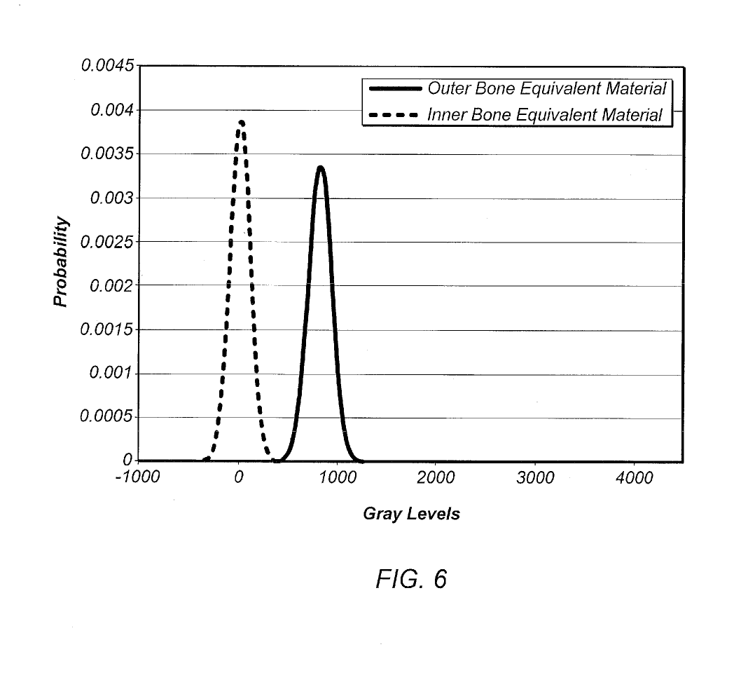

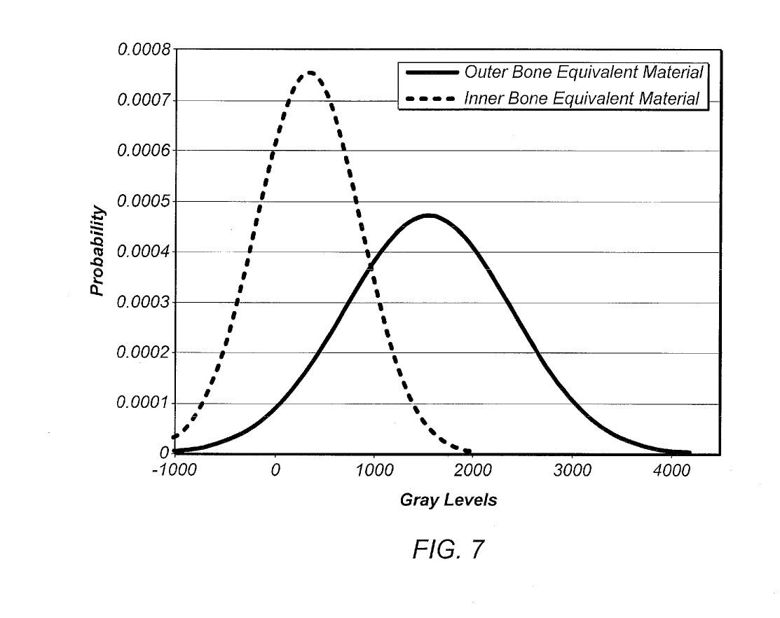

While the accuracy by which CT numbers in HU can be calculated from CBCT grey levels is very encouraging, the method utilized in this study is tedious and laborious. The strong dependence of HU on beam energy resulted in a wide variation over the patient populations, making it very difficult to distinguish the values corresponding to the various tissues from one another. FIG. 6 shows the original distribution of grey levels on the Planmeca ProMax 3D for the two bone equivalent materials in the intra-oral reference object. While the grey level values are not realistic Hounsfield Units it is at least possible to distinguish the two kinds of bone from one another. FIG. 7 shows the distribution of “corrected” grey levels. While the average values for the two materials are now realistic Hounsfield Units, the variability of effective energies results in such a tremendous overlap between the values of the two materials that it is difficult to distinguish one from another.

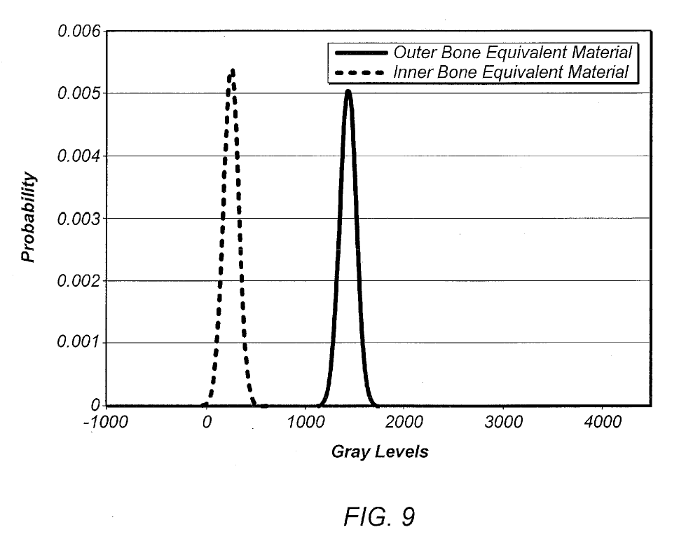

To simplify the method and reduce the overlap between the two materials, linear regressions of CT number in HU as a function of grey level were performed at several arbitrary effective energies within the range of effective energies found in the first method. The results are plotted in FIG. 8. It will noted that an excellent linear fit was obtained at each and every energy. When the same energy is used for all of the patient data, not only are reasonable HU values obtained but the distributions are much less dispersed making it possible to clearly distinguish the two kinds of bone equivalent materials as shown in FIG. 9. Using this method, it is now possible to standardize the results of the two different scanners at the same energy. Table 6 shows the averaged data from 29 patients scanned in both the Planmeca ProMax 3D and the Asahi Alphard 3030 standardized as HU at 70 keV.

TABLE 6
Average Values for Asahi Alphard 3030 and
ProMax 3D Using a Standardized HU at 70 keV

| | Asahi Alphard 3030 | Planmeca ProMax 3D |
|---|---|---|
| | Calculated HU at 70 keV | Calculated HU at 70 keV |
| Aluminum | 2237.4 | 2247.6 |
| Outer Bone | 1476.2 | 1422.7 |
| Equivalent | | |
| Inner Bone | 254.9 | 251.0 |
| Equivalent | | |
| PMMA | 80.4 | 95.2 |
| Water | 42.4 | 62.6 |
| Equivalent | | |

The results have demonstrated that the grey levels taken from CBCT scans can be utilized to derive HU in a clinical environment. This capability along with the decreased patient radiation exposure, ease of access, greater resolution than medical CT and affordability should solidify CBCT as the imaging modality of choice in dental implant placement.

### Embodiments of the Invention

A device for use in a computed tomography system comprising: a body; and one or more radiographic reference objects coupled to, or disposed in or on the body; wherein the body is positionable in a mouth of a human. The computed tomography system may be a cone beam computed tomography system or a micro computed tomography system. The device may comprise at least two radiographic reference objects having different densities. The body may comprise a polymeric resin. The radiographic reference objects may comprise two or more of: an adipose equivalent material; a water equivalent material; a muscle equivalent material; an inner (cancellous) bone equivalent material; a hard bone (cortical) equivalent material; and a material having a density greater than the density of a hard bone (cortical) equivalent material. The radiographic reference objects may be in the form of cubes having a side length of less than about 30 mm. The device may comprise a plurality of radiographic reference objects, and wherein the radiographic reference objects are positioned next to each other in a line, wherein the radiographic reference objects are placed in order of increasing density, wherein the least dense radiographic reference object is placed at one end of the line of radiographic reference objects and the most dense of the radiographic reference objects is placed at the opposite end of the line of radiographic reference objects. The one or more radiographic reference objects may be disposed in the body, wherein the body comprises a polymer resin that completely surrounds the one or more radiographic reference objects. The body may be removably couplable to a mouthpiece. The body may be in the form of a mouthpiece. The device may have a size that allows the device to be positioned inside a human mouth.

In an embodiment, a mouthpiece comprises: a mouthpiece body; and a radiographic device coupled to the mouthpiece body, wherein the radiographic device comprises: a radiographic device body; and one or more radiographic reference objects coupled to, or disposed in or on the body; wherein the mouthpiece body is positionable in a mouth of a human subject. The radiographic device of the mouthpiece may be coupled to the mouthpiece such that the radiographic device is disposed outside of the human subject's teeth when the mouthpiece is placed in a human mouth. The radiographic device of the mouthpiece may be coupled to the mouthpiece such that the radiographic device is disposed inside of the human subject's mouth when the mouthpiece is placed in a human mouth. The radiographic device of the mouthpiece may be coupled to the mouthpiece such that the radiographic device is disposed inside of the human subject's mouth, above the occlusion area of the teeth, when the mouthpiece is placed in a human mouth. The radiographic device may be removably coupled to the mouthpiece body. The mouthpiece body may comprise a coupling element that mates with a complementary coupling element of the radiographic device to couple the radiographic device to the mouthpiece body.

The radiographic device of the mouthpiece may comprise at least two radiographic reference objects having different densities. The body of the radiographic device of the mouthpiece may comprise a polymeric resin. The radiographic reference objects may comprise two or more of: an adipose equivalent material; a water equivalent material; a muscle equivalent material; an inner (cancellous) bone equivalent material; a hard bone (cortical) equivalent material; and a material having a density greater than the density of a hard bone (cortical) equivalent material. The radiographic reference objects may be in the form of cubes having a side length of less than about 30 mm. The device may comprise a plurality of radiographic reference objects, and wherein the radiographic reference objects are positioned next to each other in a line, wherein the radiographic reference objects are placed in order of increasing density, wherein the least dense radiographic reference object is placed at one end of the line of radiographic reference objects and the most dense of the radiographic reference objects is placed at the opposite end of the line of radiographic reference objects. The one or more radiographic reference objects may be disposed in the body, wherein the body comprises a polymer resin that completely surrounds the one or more radiographic reference objects.

A method of creating an image of a subject using a computed tomography system comprises: placing a radiographic device (as described in the above embodiments) proximate to a portion of a human subject, wherein the radiographic device comprises: a body; and two or more radiographic reference objects coupled to, or disposed in or on the body; collecting one or more x-ray scans using a computed tomography system, wherein at least a portion of the collected x-ray scans comprise: (a) at least a portion of the human subject; and (b) at least a portion of the radiographic device positioned proximate to the human subject; producing an image from data collected from the x-ray scans; correlating grey levels of the produced image to the attenuation coefficients of tissues and/or bones depicted in the produced image, wherein the correlation is based on measured grey levels of one or more radiographic reference objects of the radiographic device. Correlating grey levels of the produced image to the attenuation coefficients of tissues and/or bones, in one embodiment, comprises: selecting a first effective energy of the x-ray beam, based, in part, on the selected peak keV used to collect the scans; determining a function that correlates the grey levels measured for two or more of the radiographic reference objects to the attenuation coefficients of the two or more radiographic reference objects at the selected effective energy; selecting one or more additional effective energies of the x-ray beam, based, in part, on the selected peak keV used to collect the scans; determining a function for each selected additional effective energy that correlates the grey levels measured for two or more of the radiographic reference objects to the attenuation coefficients of the two or more radiographic reference objects at the selected additional effective energy; analyzing the accuracy of two or more of the determined functions to correlate the grey levels to the known attenuation coefficients; selecting an accurate function for correlating the grey levels to the known attenuation coefficients; determining CT numbers for tissues and/or bones depicted in the produced image using the selected accurate function. The correlation may be a linear function that relates measured grey levels to attenuation coefficients. In an embodiment, determining the CT number comprises: determining the attenuation coefficient of a tissues and/or bones depicted in the produced image using the selected accurate function; and calculating the CT number using the determined attenuation coefficient. Calculating the CT number may comprise calculating Hounsfield units for tissues and/or bones depicted in the produced image.

Correlating grey levels of the produced image to the attenuation coefficient of tissues and/or bones, in another embodiment, comprises: selecting an effective energy of the x-ray beam, based, in part, on the selected peak keV used to collect the scans; determining a function that correlates the grey levels measured for two or more of the radiographic reference objects to the attenuation coefficients of the two or more radiographic reference objects at the selected effective energy; determining CT numbers for tissues and/or bones depicted in the produced image using the determined function. Determining the CT number may comprise: determining the attenuation coefficient of a tissue or bone depicted in the produced image using the determined function; and calculating the CT number using the determined attenuation coefficient. Calculating the CT number comprises calculating Hounsfield units for tissues and/or bones depicted in the produced image.

In the method, the produced image comprises one of more indicators of the CT number of one or more tissues and/or bones depicted in the image, wherein the CT number of the tissues in the images are determined using the correlation. The one or more indicators of the CT number of one or more tissues and/or bones depicted in the image comprises one or more colored regions, wherein each colored region corresponds to a predetermined density range. In some embodiments, the produced image comprises one of more indicators of the Hounsfield units of one or more tissues and/or bones depicted in the image, wherein the Hounsfield units of the tissues in the images are determined using the correlation.

In an alternate method, a method of creating an image of a subject using a computed tomography system, comprises: placing a radiographic device proximate to a portion of a human subject, wherein the radiographic device comprises: a body; and two or more radiographic reference objects coupled to, or disposed in or on the body; collecting one or more x-ray scans using a computed tomography system, wherein at least a portion of the collected x-ray scans comprise: (a) at least a portion of the human subject; and (b) at least a portion of the radiographic device positioned proximate to the human subject; producing an image from the data collected from the x-ray scans; correlating grey levels of the produced image to the CT number of tissues and/or bones depicted in the produced image, wherein the correlation is based on measured grey levels of one or more radiographic reference objects of the radiographic device.

In the alternate method, correlating grey levels of the produced image to the CT numbers of tissues and/or bones comprises: selecting a first effective energy of the x-ray beam, based, in part, on the selected peak keV used to collect the images; determining a function the correlates the grey levels measured for two or more of the radiographic reference objects to the CT numbers of the two or more radiographic reference objects at the selected effective energy; selecting one or more additional effective energies of the x-ray beam, based, in part, on the selected peak keV used to collect the images; determining a function for each selected additional effective energy that correlates the grey levels measured for two or more of the radiographic reference objects to the CT numbers of the two or more radiographic reference objects at the selected additional effective energy; analyzing the accuracy of two or more of the determined functions to correlate the grey levels to the known attenuation coefficients; selecting an accurate function for correlating the grey levels to the known CT numbers; and determining CT numbers for tissues and/or bones depicted in the produced image using the selected accurate function. In an embodiment, the CT numbers are in Hounsfield units.

In the alternate method, correlating grey levels of the produced image to the CT numbers of tissues and/or bones may alternatively comprise: selecting an effective energy of the x-ray beam, based, in part, on the selected peak keV used to collect the images; determining a function the correlates the grey levels measured for two or more of the radiographic reference objects to the CT numbers of the two or more radiographic reference objects at the selected effective energy; determining CT numbers for tissues and/or bones depicted in the produced images using the determined function. In an embodiment, the CT numbers are in Hounsfield units.

In the alternative method, the produced image may comprise one of more indicators of the CT number of one or more tissues and/or bones depicted in the image, wherein the CT number of the tissues and/or bones in the image are determined using the correlation. In an embodiment, the one or more indicators of the CT number of one or more tissues and/or bones depicted in the image comprises one or more colored regions, wherein each colored region corresponds to a predetermined density range. In an embodiment, the produced image comprises one of more indicators of the Hounsfield units of one or more tissues and/or bones depicted in the image, wherein the Hounsfield units of the tissues and/or bones in the image are determined using the correlation. The correlation may be a linear function that relates measured grey levels to attenuation coefficients. The radiographic device used may be a radiographic device as described in any embodiments herein.

In an embodiment, a computed tomography system comprises: an x-ray device; and a processor coupled to the x-ray device, wherein the processor is operable to execute program instructions, and wherein the program instructions are operable to perform the method comprising: collecting one or more x-ray scans using the computed tomography system, wherein at least a portion of the collected x-ray scans comprise: (a) at least a portion of the human subject; and (b) at least a portion of a radiographic device positioned proximate to the human subject, wherein the radiographic device comprises: a body; and one or more radiographic reference objects coupled to, or disposed in or on the body; and producing an image from data collected from the x-ray scans; correlating grey levels of the produced image to the attenuation coefficients of tissues and/or bones depicted in the produced image, wherein the correlation is based on measured grey levels of one or more radiographic reference objects of the radiographic device. The system may be used to create an image of a subject using a computed tomography system using a method as described in any embodiments herein.

In an alternate embodiment, a computed tomography system comprises: an x-ray device; and a processor coupled to the x-ray device, wherein the processor is operable to execute program instructions, and wherein the program instructions are operable to perform the method comprising: collecting one or more x-ray scans using the computed tomography system, wherein at least a portion of the collected x-ray scans comprise: (a) at least a portion of the human subject; and (b) at least a portion of a radiographic device positioned proximate to the human subject, wherein the radiographic device comprises: a body; and one or more radiographic reference objects coupled to, or disposed in or on the body; and producing an image from data collected from the x-ray scans; correlating grey levels of the produced image to the CT numbers of tissues and/or bones depicted in the produced image, wherein the correlation is based on measured grey levels of one or more radiographic reference objects of the radiographic device. The system may be used to create an image of a subject using a computed tomography system using a method as described in any embodiments herein.

The methods described in any embodiments herein may be embodied on a tangible, computer readable medium comprising program instructions. The program instructions are computer-executable to implement the method of creating an image of a subject using a computed tomography system, using a method as described in any embodiments herein.

In an embodiment, a method of creating an image of a subject using a computed tomography system, the method comprising: collecting one or more x-ray scans using a computed tomography system, wherein at least a portion of the collected x-ray scans comprise: (a) at least a portion of a human subject's teeth; and (b) at least a portion of muscle tissue of a human subject; producing an image from data collected from the x-ray scans; correlating grey levels of the produced image to the attenuation coefficients of tissues and/or bones depicted in the produced image, wherein the correlation is based on measured grey levels of one or more portions of the human subject's teeth and the grey levels of one or more portions of muscle tissue of the human subject.

In an embodiment, a method of creating an image of a subject using a computed tomography system comprises: collecting one or more x-ray scans using a computed tomography system, wherein at least a portion of the collected x-ray images scans comprise: (a) at least a portion of a human subject's teeth; and (b) at least a portion of muscle tissue of a human subject; producing an image from data collected from the x-ray scans; correlating grey levels of the produced image to the CT numbers of tissues and/or bones depicted in the produced image, wherein the correlation is based on measured grey levels of one or more portions of the human subject's teeth and the grey levels of one or more portions of muscle tissue of the human subject.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A device for use in a computed tomography system comprising:
a body; and
one or more radiographic reference objects coupled to, or disposed in or on the body;
wherein the body is positionable in a mouth of a human.

2. The device of claim 1, wherein the device comprises at least two radiographic reference objects having different densities.

3. The device of claim 1, wherein the body comprises a polymeric resin.

4. The device of claim 1, wherein the radiographic reference objects comprise two or more of: an adipose equivalent material; a water equivalent material; a muscle equivalent material; an inner (cancellous) bone equivalent material; a hard bone (cortical) equivalent material; and a material having a density greater than the density of a hard bone (cortical) equivalent material.

5. The device of claim 1, wherein the device comprises a plurality of radiographic reference objects, and wherein the radiographic reference objects are positioned next to each other in a line, wherein the radiographic reference objects are placed in order of increasing density, wherein the least dense radiographic reference object is placed at one end of the line of radiographic reference objects and the most dense of the radiographic reference objects is placed at the opposite end of the line of radiographic reference objects.

6. The device of claim 1, wherein the one or more radiographic reference objects are disposed in the body, and wherein the body comprise a polymer resin that completely surrounds the one or more radiographic reference objects.

7. A mouthpiece comprising: a mouthpiece body; and a radiographic device coupled to the mouthpiece body, wherein the mouthpiece body is positionable in a mouth of a human subject; and wherein the radiographic device comprises: a body and one or more radiographic reference objects coupled to, or disposed in or on the body; wherein the body is positionable in a mouth of a human.

8. The mouthpiece of claim 7, wherein the radiographic device is removably coupled to the mouthpiece body.

9. A method of creating an image of a subject using a computed tomography system, the method comprising:
placing a radiographic device proximate to a portion of a human subject, wherein the radiographic device comprises: a body and one or more radiographic reference objects coupled to, or disposed in or on the body; wherein the body is positionable in a mouth of a human;
collecting one or more x-ray scans using a computed tomography system, wherein at least a portion of the collected x-ray scans comprise: (a) at least a portion of the human subject; and (b) at least a portion of the radiographic device positioned proximate to the human subject;
producing an image from data collected from the x-ray scans;
correlating grey levels of the produced image to the attenuation coefficients and/or the CT number of tissues and/or bones depicted in the produced image, wherein the correlation is based on measured grey levels of one or more radiographic reference objects of the radiographic device.

10. The method of claim 9, wherein correlating grey levels of the produced image to the attenuation coefficients and/or CT numbers of tissues and/or bones comprises:
selecting a first effective energy of the x-ray beam, based, in part, on the selected peak keV used to collect the scans;
determining a function that correlates the grey levels measured for two or more of the radiographic reference objects to the attenuation coefficients of the two or more radiographic reference objects at the selected effective energy;
selecting one or more additional effective energies of the x-ray beam, based, in part, on the selected peak keV used to collect the scans;
determining a function for each selected additional effective energy that correlates the grey levels measured for two or more of the radiographic reference objects to the attenuation coefficients of the two or more radiographic reference objects at the selected additional effective energy;
analyzing the accuracy of two or more of the determined functions to correlate the grey levels to the known attenuation coefficients;
selecting an accurate function for correlating the grey levels to the known attenuation coefficients;
determining CT numbers for tissues and/or bones depicted in the produced image using the selected accurate function.

11. The method of claim 9, wherein determining the CT number comprises:
determining the attenuation coefficient of a tissues and/or bones depicted in the produced image using the selected accurate function;
calculating the CT number using the determined attenuation coefficient.

12. The method of claim 9, wherein calculating the CT number comprises calculating Hounsfield units for tissues and/or bones depicted in the produced image.

13. The method of claim 9, wherein correlating grey levels of the produced image to the attenuation coefficients and/or CT numbers of tissues and/or bones comprises:
selecting an effective energy of the x-ray beam, based, in part, on the selected peak keV used to collect the scans;
determining a function that correlates the grey levels measured for two or more of the radiographic reference objects to the attenuation coefficients of the two or more radiographic reference objects at the selected effective energy;
determining CT numbers for tissues and/or bones depicted in the produced image using the determined function.

14. The method of claim 9, wherein determining the CT number comprises:
determining the attenuation coefficient of a tissue or bone depicted in the produced image using the determined function;
calculating the CT number using the determined attenuation coefficient.

15. The method of claim 9, wherein the produced image comprises one of more indicators of the CT number of one or more tissues and/or bones depicted in the image, wherein the CT number of the tissues in the images are determined using the correlation.

16. The method of claim 9, wherein the produced image comprises one of more indicators of the Hounsfield units of one or more tissues and/or bones depicted in the image, wherein the Hounsfield units of the tissues in the images are determined using the correlation.

17. A computed tomography system comprising: an x-ray device; and a processor coupled to the x-ray device, wherein the processor is operable to execute program instructions, and wherein the program instructions are operable to perform the method comprising:
placing a radiographic device proximate to a portion of a human subject, wherein the radiographic device comprises: a body and one or more radiographic reference objects coupled to, or disposed in or on the body; wherein the body is positionable in a mouth of a human;
collecting one or more x-ray scans using a computed tomography system, wherein at least a portion of the collected x-ray scans comprise: (a) at least a portion of the human subject; and (b) at least a portion of the radiographic device positioned proximate to the human subject;
producing an image from data collected from the x-ray scans; and
correlating grey levels of the produced image to the attenuation coefficients and/or the CT number of tissues and/or bones depicted in the produced image, wherein the correlation is based on measured grey levels of one or more radiographic reference objects of the radiographic device.

18-21. (canceled)
